(19) United States
(12) Patent Application Publication
(10) Pub. No.: US 2004/0180015 A1
(43) Pub. Date: Sep. 16, 2004

(54) Long-acting disinfecting nitrous acid compositions and related processes

(76) Inventors: Robert D. Kross, Bellmore, NY (US); Lorrence H. Green, Westbury, NY (US)

Correspondence Address: COLEMAN SUDOL SAPONE, P.C., 714 COLORADO AVENUE, BRIDGE PORT, CT, 06605-1601, US

(21) Appl. No.: 10/780,435

(22) Filed: Feb. 17, 2004

Related U.S. Application Data
(63) Continuation-in-part of application No. 10/041,310, filed on Jan. 7, 2002, now abandoned.
(60) Provisional application No. 60/511,916, filed on Oct. 17, 2003.

(51) Int. Cl. A61K 7/06; A61K 31/19; A61K 33/14

(52) U.S. Cl. 424/70.1; 424/718; 514/557

(57) ABSTRACT

The invention relates to long-acting nitrous acid compositions that disinfect inanimate surfaces and animal tissues, and that can be used to treat diseases and wounds. Compositions of the invention are single-phase, metastable nitrous acid solutions that exhibit enhanced germicidal efficacy and can have an effective lifetime exceeding several years. Compositions of the invention have a wide variety of applications that include, but are not limited to, teat dips, oral rinses, instrument sterilization, and disinfection of food.

### RELATED APPLICATIONS

This application is a continuation-in-part application of U.S. application Ser. No. 10/041,310, filed Jan. 7, 2002, and also claims priority from provisional application 60/511,916, filed Oct. 17, 2003, each of which application is incorporated by reference in its entirety herein.

### FIELD OF THE INVENTION

The invention relates to long-acting and storage stable nitrous acid compositions that disinfect inanimate surfaces and animal tissues, and that can be used to treat diseases and wounds. Compositions of the invention are single-phase, metastable nitrous acid solutions that exhibit enhanced germicidal efficacy and can have an effective lifetime optimally exceeding several years. Compositions of the invention have a wide variety of applications that include, but are not limited to, teat dips, oral rinses, instrument sterilization, and disinfection of food.

### BACKGROUND OF THE INVENTION

Disinfectants in which an inactive oxyanion and a suitable proton donor are combined shortly before the product's intended use are well-known. The subsequent degradation of the resulting acid into a series of transient, cidal oxidants proves effective in killing or inactivating a broad spectrum of bacteria, yeasts, molds and viruses in a very rapid manner. Such disinfectants rely on in situ creation of metastable chlorous acid in aqueous chlorite (ClO₂⁻) solutions, under conditions where the chlorous acid, HClO₂, represents a relatively small fraction of the total chlorite ion present (typically no more than about 15%), in order to minimize the otherwise rapid degradation of the system. The antimicrobially-effective chlorous acid systems function at pH values from about 3.5 down to about 2.6. The discovery that it was the chlorous acid itself, rather than a breakdown product, i.e., the well-known germicide chlorine dioxide (ClO₂) that is formed in these solutions, was the key to the expanding use of this germicidal agent.

The protic acid source to effect this conversion is generally an organic acid (U.S. Pat. Nos. 4,986,990, 5,185,161), although inorganic acids (U.S. Patent No. RE 36,064) and even acid-inducing metal salts have been disclosed (U.S. Pat. No. 5,820,822). Acidified chlorite compositions are disclosed in U.S. Pat. Nos. 4,084,747 and 4,330,531.

A diverse range of acidified chlorite compositions and related methods are disclosed in U.S. Pat. Nos. 4,891,216 (for topical application); 4,956,184 (for genital herpes); 5,100,652 (for oral hygiene); 5,384,134 (for anti-inflammatory activity); 5,389,390 and 6,063,425 (for disinfecting poultry and other meats); 5,597,561 and 5,651,977(adherent topical disinfectants); 5,628,959 (sterilizing hemodialyzers); 5,772,985 (bovine warts); 6,096,350 (for honey bee diseases); and 6,123,966 (stabilized disinfecting compositions).

Although acidified chlorite systems are useful as disinfectants, several inherent characteristics of such systems limit their application in certain situations. The chlorite ion and the acid activator must be maintained separately until shortly before use, at which point the two components (in solution form) are combined and then rapidly applied to the substrate within hours, or even minutes, of combination. The rapid degradation of the unstable chlorous acid that is formed in the mixed solution (to chlorine dioxide, chloride and chlorate ion) requires that fresh combinations be prepared each time a disinfecting chlorous solution is needed. As a result, it is not possible to prepare a single chlorous acid system that is as shelf-stable as germicides as glutaraldehyde, iodophors, quaternary ammonium compounds, and peroxides.

The relatively strong oxidizing tendency of acidified chlorite systems, and the particularly corrosive effects of the chlorine dioxide (ClO₂) which forms upon degradation of the chlorous acid, is also disadvantageous. ClO₂ will corrode many of the metals used in the fabrication of medical and dental equipment, as well as the metals associated with equipment used to dispense the solutions for such applications as the commercial disinfection of poultry, meats and agricultural commodities. A further detriment of the acidified chlorite systems is the noxiousness of the ClO₂ gas, for which OSHA has listed a very low permissible concentration in the air to which workers may be exposed for an 8 hour period. That level, 0.1 parts per million in the air, is 10 times lower, for example, than for chlorine, for which OSHA has listed a maximum permissible level of 1.0 ppm over an 8-hour period.

PCT Patent Application WO 95/22335 discloses the use of acidified nitrite as an antimicrobial agent. In the disinfectant systems of PCT Patent Application WO 95/22335, acidified nitrites form nitrous acid to release oxides of nitrogen to destroy microorganisms, in a manner analogous to the mechanism by which chlorous acid is caused to release the corresponding oxide (chlorine dioxide) which acts to destroy microorganisms. Such activated oxyanion solutions, whether chlorous acid or nitrous acid based, do not have the inherent ability to survive for protracted time periods and provide germicidal action. Further, disinfectant systems of PCT Patent Application WO 95/22335 function by the release of destructive oxides of nitrogen. The high instability of nitrogen oxides in the presence of air (i.e. oxygen) would militate against any long-term survivability of such solutions, unless perhaps stored in oxygen-free environments.

Applicants' U.S. Provisional Patent Application No. 60/511/916 discloses that acidified nitrite teat dip compositions can be used in a wider range of germicidal formulations, including teat care products.

The need continues to exist, however, for stable or metastable nitrous acid systems capable of providing a high level of germicidal activity over the course of a number of years. Such a single-phase germicide could prove more effective than highly regarded, dual-phase acidified chlorite systems and should exhibit benefits including reduced corrosion, reduced tissue decolorization, improved safety and, of major importance, the “mix-and-use” requirement of chlorous acid systems.

### OBJECTS OF THE INVENTION

It is an object of the present invention to provide antimicrobial nitrous acid solutions which can maintain their germicidal activity for several years after preparation.

It is a further object of the invention to control the antimicrobial activity of these long-acting solutions by modifying their initial acidity and thus the degree of conversion of nitrite ion to nitrous acid.

It is yet a further object of the invention to provide compositions based upon nitrous acid which, shortly after preparation and for extended periods thereafter, exhibit a broad spectrum of antimicrobial action.

It is still another object of the invention to provide long-lasting nitrous acid solutions which continuously exhibit significant antimicrobial activity with minimum corrosion of medical, dental and other metallic substrates which require disinfection.

It is an additional object of the invention to provide long-lasting nitrous acid solutions which exhibit antimicrobial activity with minimum decolorization and bleaching of animal tissue substrates.

### SUMMARY OF THE INVENTION

The invention provides novel single-phase compositions comprising a liquid or gel comprising nitrous acid and an alpha hydroxyl acid, wherein:

(a) the pH of the composition either remains relatively constant at a value of around 3.7 or lower, or decreases from an initial value of around 3.7 to as low as about a value of around 2.5 over a period of at least about two days, preferably between about two to about five days;

(b) the molar percentage of nitrite ion in the composition in the form of nitrous acid is greater than about 35% but less than about 95% of the total nitrite ions present in the composition; and

(c) the composition exhibits cidal activity against microorganisms for a period of at least several months after formulation.

The germicidal efficacy of compositions of the invention is dependent on nitrous acid in the compositions, not on the generation of nitric oxide following the acidification of nitrite salts.

Compositions of the invention have a wide variety of applications that include, but are not limited to, teat dips, oral rinses, instrument sterilization, and food disinfection.

In preferred embodiments, compositions of the invention are used in applications including sterilization of medical instrumentation and in teat dips.

Nitrous acid compositions of the invention exhibit prolonged antimicrobial efficacy for extended periods, e.g., several years after formulation. The compositions comprise an aqueous or gel solution containing a suitable amount of a protic acid, or a material inducing an acidic environment therein, and a suitable amount of a metal nitrite. The nitrite ion concentration in the composition, in the form of nitrous acid, is greater than about 35% but no more than about 95%, by weight, of the total amount of nitrite ion concentration in the composition.

In a preferred embodiment, the invention provides a disinfecting liquid composition comprising a nitrous acid generating compound and an organic acid that lowers the pH of the composition to below 3.75. The preferred organic acid is an alpha hydroxy acid of the formula (I):

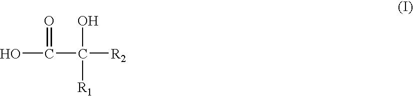

wherein R₁ and R₂ may be the same or different and may be selected from the group consisting of hydrogen, methyl, —CH₂ COOH, —CH₂ COO⁻, —CH₂ OH, —CHOHCOOH, —C₆H₅, and —CH₂ C₆H₅. The pKa of the organic acid may be from about 2.8 to about 4.8.

In another preferred embodiment, compositions of the invention comprise a compound comprising an amount of phosphoric acid with a pKa of about 2.15 that is sufficient to lower the pH of the composition to less than about 3.75.

In another aspect, the present invention provides processes for disinfecting a substrate using the compositions described above. These processes comprise applying the compositions described above to a substrate, from about 5 minutes after preparation of such compositions up through at least several years after their preparation, in order to disinfect the substrate.

In yet another aspect, the present invention provides a process for preparing disinfecting compositions and for disinfecting a surface using the resulting nitrous acid-containing composition. The process comprises contacting a protic acid, or a solution with induced acidity, with the metal nitrite to form the disinfecting compositions, which are then allowed to equilibrate for at least about five minutes prior to use in effective amounts to disinfect a desired surface.

These and other aspects of the invention are described further in the detailed description of the invention.

### DETAILED DESCRIPTION OF THE INVENTION

As used herein, the following terms have the following respective meanings The term “nitrite” or “nitrite salt” describes a salt of nitrous acid which is readily soluble in an aqueous or gel system and which readily dissociates into nitrite anion and counterion (generally, metal). Two particularly preferred salts of nitrites for use in the present invention include sodium nitrite and potassium nitrite, although a number of other nitrite salts may also be used in the present invention. The term “nitrite” describes the form in which an amount of a water soluble salt of nitrous acid either in dry or liquid state (preferably, as an aqueous solution) is added to the acid. In general, the nitrite is added to the acid and preferably, both the nitrite and the acid are mixed together in an aqueous solution to which has been added effective amounts of additives such as surfactants, coloring agents, chelating agents and gelling agents, as otherwise described herein. Metal nitrite salts are preferred for use in the present invention.

The term “nitrite ion” describes the nitrite anion of a nitrite salt. Where the term “nitrite ion” is described in amounts in a given aqueous composition, it is the amount or concentration of the anion which is being referenced, not the amount of total salt concentration which generally contains both a nitrite anion and a metal cation.

“Acid” describes protic acids, i.e., acids that release hydrogen ions in solution. Acids used in the present invention include strong inorganic acids such as hydrochloric, sulfuric, and nitric acid; alkylsulfonic acid and benzenesulfonic acid, among other organic sulfonic acids, which, depending upon the end-use of the composition, may be preferably included as dilute acid; organic acids such as citric, fumaric, glycolic, lactic, malic, maleic, tartaric acid, salicylic, citric, propionic, acetic and mandelic, among others, including ethylenediaminetetraacetic acid (EDTA, as the free acid or the monosodium salt), among others; and inorganic acids such as sodium and potassium bisulfate (NaHSO₄ and KHSO₄) and phosphoric acid, among numerous others. It is noted that numerous additional acids may also be used in the present invention. In its broadest aspect, compositions according to the present invention may make use of virtually any acid, to the extent that it provides an initial pH, which when the nitrite-containing part and the acid-containing part are combined produce nitrous acid in amounts effective for the intended purpose. One of ordinary skill will be able to readily determine the type and amount of acid to be used for a particular application.

“Material inducing an acidic environment therein” describes a material, which, when added to compositions according to the present invention, produces an acidic environmental as a consequence of the interaction of the material with an aqueous solution. Such materials include for example, various Lewis Acids, numerous acid inducing metal salts, including, for example, aluminum cations, gadolinium cations, vanadium cations, zirconium cations, zinc cation, more specifically and preferably, for example, aluminum chlorhydroxide, aluminum acetate, aluminum ammonium sulfate, aluminum phenolsulfonate, iron, aluminum, gadolinium and vanadium chlorides, zirconium oxychloride; zinc, cadmium and magnesium salts of chloride, nitrate, sulfate, perchlorate, acetate, citrate, and lactate, among others.

The term “substrate” as used in the instant specification is intended to cover any type of surface or carrier which could provide a locus for the accumulation of germs (bacteria, yeasts, molds, viruses, i.e., all types of infectious agents). Obvious examples embrace medical and dental surfaces, including endoscopes, surgical and dental equipment, pharmaceutical and food plants, foods, food containers, human and animal skin and tissues, body fluids and mucous membranes, home areas such as in kitchens, as well as bathroom appliances, food surfaces, sanitation equipment, etc.

The term “effective amount” is used to describe that amount of a composition, an individual component or a material which is included in compositions according to the present invention in order to produce an intended effect. For example, in the case of an effective amount of an acid, an effective amount is that amount which is included to produce a sufficiently acidic medium to produce nitrous acid in combination with a nitrite salt wherein the nitrite ion concentration in the composition, in the form of nitrous acid, is greater than about 35% but no more than about 95%, by weight, of the total molar amount of nitrite ion in the composition.

An effective amount of nitrite or a nitrite salt is that amount which is effective to produce a desired concentration of nitrous acid after mixing with an appropriate and effective amount of an acid. In the case of a gelling agent, an effective amount of that component is that amount which is effective to gel a final composition (i.e., produce a viscous composition). One of ordinary skill will be able to readily determine effective amounts of components or compositions for use to provide an intended effect.

The term “gelling agent” describes a compound or composition which is added to compositions of the invention in order to increase the viscosity of the composition. Gelling agents which are used in the present invention may be added to the nitrite-containing part (A) or the acid-containing part (B) in amounts effective to gel the solution to which these compounds have been added. Gelling agents for use in the present invention include polysaccharides extracted from legume seeds, such as the galactomannans, including guar gum and locust bean (carob) gum. Other gelling agents include high molecular weight polyoxyalkylene crosslinked acrylic polymers as well as the highly preferred cellulosics such as hydroxymethyl cellulose, hydroxypropyl cellulose, hydroxyethyl cellulose, methyl cellulose, methylpropyl cellulose, among others, including high molecular weight polyethylene glycols, polyacrylamide and polyacrylamide sulfonates, and crosslinked polyvinylpyrrolidones, among others.

As described above, the present invention is directed to a nitrous acid-containing composition for disinfecting a substrate. The composition comprises an aqueous solution containing a suitable amount of hydrogen ions derived from either a protic acid or a material which induces an acidic environment therein such as an acid-inducing salt, and a suitable amount of nitrous acid derived from partial acidification of a metal nitrite such as sodium nitrite. Compositions according to the present invention are preferably produced by adding a metal nitrite (either as a dry material or in solution) to an acidic solution. The concentration of hydrogen ion-generating species is such that the amount of nitrite ion in the form of nitrous acid is greater than about 35% but no more than about 95% by weight of the total nitrite ion in the solution. Preferably, the amount of nitrite in the form of nitrous acid is no more than about 85% by weight of the total nitrite ion concentration in solution.

The percent by weight of nitrite and nitrous acid may be calculated from the ionization constant of nitrous acid and the amount of hydrogen ion in solution produced by partial ionization of the protic acid, or calculated from the pH of a salt-induced acid solution. The hydrogen ion concentration, [H⁺], in a solution of a protic acid, HA, of known molar concentration and whose ionization constant is Ka, may be calculated from the following relationship:

$$Ka = \frac{[ {H}^{+} ] [ {A}^{-} ]}{[ HA ]}$$

For solutions where the acidity is induced by the presence of a particular salt, [H⁺] is determined by measurement of the solution's pH and calculation from the negative antilog of that value.

The above relationship may be applied to calculate the relative nitrite and nitrous acid concentrations, where the ionization constant for nitrous acid is 4.5×10⁻⁴. That is:

$$4.5 \times {10}^{- 4} = \frac{[ {H}^{+} ] [ {NO}_{2}^{-} ]}{[ {HNO}_{2} ]}$$

where the hydrogen ion concentration, [H⁺], is the quantity readily determined by ionization of the known amount of the protic acid, HA. This calculation is well known to those skilled in this art. For the nitrous acid/nitrite system, the following Table 1 illustrates representative percentages of both species over a pH range which provides high to low amounts of nitrous acid, as derived from nitrite.

TABLE 1
Percentage of Nitrite as Nitrous Acid
at Varying pH Values

| [symbol] | | Nitrous Acid % | Nitrite % |
|---|---|---|---|
| | 1.5 | 98.4 | 1.6 |
| | 2.0 | 95.2 | 4.8 |
| | 2.3 | 90.9 | 9.1 |
| | 2.6 | 83.3 | 16.7 |
| | 2.8 | 76.0 | 24.0 |
| | 3.0 | 66.7 | 33.3 |
| | 3.3 | 50.0 | 50.0 |
| | 3.5 | 38.8 | 61.2 |
| | 4.0 | 16.6 | 83.4 |
| | 4.5 | 6.0 | 94.0 |
| | 5.0 | 2.0 | 98.0 |

Aqueous solutions of nitrous acid are generally regarded to be unstable, and decompose according to the following equation. Instability increases with increased absolute and relative molar concentrations of the HONO, and with increasing heat:

3HNO₂(aq)<--->H⁺(aq)+2NO(g)+NO₃⁻(aq)+H₂O(l) Equation 1

The reaction is a combination of the two half-reactions, as follows:

2{HNO₂+H⁺+e⁻<--->NO+H₂O} Equation 2

$$\sum = \frac{{HNO}_{2} + {H}_{2} O ↔ {NO}_{3}^{-} + 3 {H}^{+} + 2 e -}{ 3 {HNO}_{2} ( aq ) ↔ {H}^{+} ( aq ) + \\ 2 NO ( g ) + {NO}_{3}^{-} ( aq ) + {H}_{2} O ( 1 )} \qquad Equation 3$$

In addition to the concentration-dependent degradation of nitrous acid, as shown above, nitrous acid will also act as an oxidizing agent in the presence of oxidizable materials, such as microorganisms, according to the first half-cell reaction above (Equation 2), with a redox potential ε°=1.00 volts. Accordingly nitrous acid systems are quite destructive of all classes of microorganisms which are susceptible to oxidation, including bacteria, yeasts, molds and viruses. This destruction is well known for other non-specific oxidizing germicides such as bleach (hypochlorous acid), chlorous acid, chlorine dioxide, and iodine.

We have discovered that acidified nitrite solutions, upon standing, will generally become either more acidic or less acidic in rough proportion to the pH of the solution, and thus the relative amount of nitrous acid with respect to nitrite ion. At concentrations of nitrous acid with respect to total nitrite that are greater than about 30%, stored solutions will generally become more acidic. At relative concentrations of nitrous acid lower than that, the stored solutions will generally become more alkaline (i.e. less acidic). In one set of experiments the break-point with respect to greater or lesser acid formation occurred at ˜pH 3.7, (i.e., corresponding to an acidity at which about 30% of the total nitrite ion present exists in the nitrous acid form), and where the total molar concentration of nitrite [ionic and acid-form] was 0.045M/liter. The data were as follows:

| pH at T = 0 | pH at T = 30 days |
|---|---|
| 2.94 | 2.30 |
| 3.12 | 2.50 |
| 3.35 | 3.25 |
| 3.54 | 3.15 |
| 3.75 | 3.92 |
| 3.90 | 4.35 |

The first solution, at pH 2.94, with a relative nitrous acid level of about 70% (see Table 1), increased in acidity to 2.30, a pH drop of 0.64 units, whereas the last solution, at pH 3.90, and a relative nitrous acid level of about 20%, increased in pH by 0.45 units. The quantity of acid required to reduce the pH in the first solution is, of course, much greater than for the last solution, in large measure because of the logarithmic basis for the pH scale.

While not wishing to be bound by any theory or in any way intending the limit the scope of the instant invention, the increase or decrease of solution pH is believed to be related to the corresponding contributions of half-cell Equations 2) and 3) above, the former reducing the H⁺ present in solution (i.e. raising the pH) and the latter contributing H⁺ to the medium, and thereby lowering the pH. There may be some involvement of the organic acidifier, which in this experiment was malic acid, in the overall reaction characteristic of the particular combination of nitrite and acid concentrations in this set of solutions.

However it is evident from the above-referenced experiment that it is feasible to adjust the concentrations of nitrite and acid in a preferred composition of this invention such that the solution is stabilized or slowly reduces in pH value over a prolonged period of time, and is capable of being stored as a pre-mixed one-part composition. In fact, it has now been determined that the pH values of nitrous acid solutions which become lower over time (i.e., show decreasing pH values) apparently level off over time, when the total metal nitrite concentrations of the nitrous acid compositions are below about 1.0%. Thus, one may not only formulate a nitrous acid solution that maintains a relatively constant pH value over an extended time, and thereby provide continued germicidal activity, but one can judiciously formulate a nitrous acid composition which decreases in pH over time, prior to leveling off, and which continues to provide equivalent or superior antimicrobial activity during that entire period. This aspect of the invention is described further in Examples 3, 6, 7, and 8 herein.

Excess nitrite ions, at levels consistent with this invention, may also suppress degradation of the claimed compositons.

In the acidified NO₂⁻/HONO system, i.e. NO₂⁻+H⁺<--->HONO, where only a fraction of the NO₂⁻ has been converted to the HONO germicidal source, when the latter has been depleted or consumed in solution, additional HONO forms from the residual NO₂⁻. The greater the degree of initial conversion, as a function of the system's pH and/or microbially-triggered degradation, the lower the reservoir of NO₂⁻ and the lower the absolute amount of HONO that can subsequently form. But the greater the initial HONO, the greater the potential cidal activity that is available for the system initially. And, apparently, the greater is the potential for the HONO in the system to drop in pH and have increasing cidal activity, in its stored solutions. This surprising finding is inconsistent with past teachings, which stress the increasing instability of nitrous acid solutions with increasing relative amounts in solution (i.e. at more and more acidic concentrations).

Further, the lower the initial HONO the greater the reservoir of NO₂⁻ ion, and the lower the germicidal action. This surprisingly does not affect the stability of the nitrous acid system, unless the initial pH is above about 3.75, at which point the solution pH slowly increases to form lower and lower nitrous acid/nitrite ion ratios and insignificant germicidal potential. The use of acid activating systems which provide a reservoir of [H⁺] ions, such as α-hydroxy acids, or phosphoric acid, which are not fully ionized initially, allows for additional [H⁺] ions to combine with the NO₂⁻ in the reservoir. As nitrous acid and its associated cidal species are consumed by germicidal activity, more of the HONO can form from this acid reservoir. Of course, for applications where only shorter-term activity is needed, even mineral acids can serve as the proton source. This is accomplished by selecting acid concentrations where the pH of the system is such that the molar percentage of nitrite ion in the form of nitrous acid is greater than about 35% but less than about 95% of the total nitrite ion amount.

In certain embodiments of the invention, the nitrous acid generating composition comprises less than about 1.0, preferably about 0.01 to about 0.75, more preferably 0.03 to about 0.70, and even more preferably from about 0.05 to about 0.50 percent by weight of metal nitrite, and a suitable amount of an acid having a pKa of from about 2.1 to about 4.8, to reduce to pH of the composition to about 3.7 or less. The pH of this composition is generally less than about 3.7, and typically from about 2.5 to about 3.6 within this range. At metal nitrite levels higher than about 0.7%, the concentration of nitrous acid formed upon admixture of a protic acid, in the typical pH range specified, may be in excess of that required for the formation of a metastable nitrous acid germicidal solution. These higher concentrations of nitrous acid could promote too rapid a formation of nitric oxide, and nitrogen dioxide therefrom, through the further oxidation of nitric acid, viz.

3HNO₂<--->H⁺+2NO+NO₃⁻+H₂O; then: 2NO+O₂(air)<--->2NO₂ [nitrogen dioxide]

Such solutions would not be appropriate for use in applications where extended lifetimes of at least one day are generally preferred.

Any protic acid, or acidic environment otherwise created, may be used in the present invention so long as the nitrite ion concentration limits described above are met. Suitable protic acids include such inorganic acids as phosphoric acid, and such α-hydroxy organic acids as citric, malic, lactic, tartaric, glycolic, mandelic or other structurally similar acids as described in Formula 1 hereinabove and hereinbelow, for convenience. The pKa of these organic acids may be generally from about 2.8 to about 4.2, and preferably from about 3.0 to about 4.0. Also suitable are such other acids as salicylic acid and acetic acid.

The amount of acid, or acid-inducing salt, used in these compositions should be sufficient to lower the pH of the composition to less than about 3.7, and preferably from about 2.5 to about 3.6. The range of compositions is, of course, very broad, since useful acids range from the weak, such as acetic acid with a pKa of 4.76 to the moderately strong, such as tartaric acid with a first pKa of 3.03 and phosphoric acid with a first pKa of 2.12. Even mineral acids may be used, where sufficiently small amounts are needed to provide the solutions of such Normality that the requisite pHs are achieved.

Other embodiments of the invention may be formulated for a specific disinfecting procedure, or as a result of a specific production method. These embodiments may contain an acid, or acid-inducing component, e.g., aluminum chloride, which is specifically suited for that procedure or production method. The acid-inducing salts are those taught in U.S. Pat. No. 5,820,822, which is incorporated herein by reference.

While any metal nitrite is useful in the present composition, the alkali and alkaline earth nitrites are preferred because they are readily soluble, readily available and inexpensive. Sodium nitrite, potassium nitrite and ammonium nitrite are preferred. Sodium nitrite is particularly preferred.

The disinfection composition may be used in conjunction with an application medium. The application medium may be any compatible medium including a thickened solution, a gel or a liquid in which water represents a sufficient enough component that the normal equilibrium of the nitrite ion and nitrous acid may exist. An aqueous application medium is preferred. The application medium may contain other additives such as chelating agents (e.g. Na₂H₂ EDTA), surfactants (e.g., alkyl aryl sulfonates such as Nacconol, and nonionic polyoxyalkylene nonylphenols such as Triton N-101), preservatives (e.g., sodium benzoate) or colors (e.g., FD&C Blue #1).

The nitrous acid solutions, in these inventive compositions, should be prepared at some time prior to their intended application, but are not to be applied directly after their preparation. Specifically they are not intended for immediate use, either by 1)- mixing the nitrite salt and the acidifier in an aqueous environment, and contacting the microbially-contaminated surface within the first minutes of their combination, or 2)-applying either the acid- or nitrite-containing component to the substrate to be disinfected followed by the other component and mixing both components on that substrate. This time restriction allows for the solution components to properly equilibrate prior to their subsequent application.

In general, once the two parts are mixed, there is an initial formation of nitrous acid followed by degradation of the nitrous acid, at a rate dependent on such factors as time, temperature, and concentration of the nitrous acid. The relative amount of nitrous acid with respect to total nitrite ion in acidified nitrite solutions will depend upon both the absolute concentration of nitrite ion and the acidity of the system, as demonstrated in Table 1 hereinabove.

When the pH of nitrous acid systems of the invention is above about 3.75 the nitrous acid solution will provide short-term cidal activity, possibly due to the freshly-generated nitric oxide. However nitrous acid solutions of the invention which have more than about 30% of their total nitrite ion in the form of nitrous acid (at about pH 3.7), are capable of sustained germicidal action up through several years following their preparation. Such solutions apparently continue to generate hydrogen ion during their aging, but it has been particularly noted that during that time such stored solutions, even in lightly-capped containers, do not result in significant further generation of nitric oxide, even while the relative level of nitrous acid increases. This is a surprising finding, but may relate to the relative low levels of nitrite salts which comprise these inventive compositions (below about 1.0%).

Example 3 demonstrates that nitrous acid solutions, derived from 0.3125% sodium nitrite, and which had been stored loosely capped for 20 days, showed comparable or greater germicidal activity than when evaluated five minutes after their preparation, when such solutions had pH values lower than about 3.7. In Example 6, teat dip compositions comprising nitrous acid solutions derived from 0.3125% sodium nitrite, had virtually the same cidal action after two (2) days as did the solutions that were 10 minutes old. In Example 7, the same teat dip composition showed uniformly excellent cidal activity, approximating 8.5 logs (>300,000,000 organisms) from Day 0 through Day 14. Finally, in Example 8, a solution that had been evaluated in Example 3, at both time 0 and after 20 days of storage, was shown to be fully active (>8.15 logs kill in 5 minutes) after 26 months (specifically 735 days) of storage under ambient conditions.

Antimicrobial action may be enhanced or extended by inclusion of a variety of agents in the nitrous acid compositions. These agents may include surface active materials, chelating agents, effervescent compounds and thickeners. These materials must have a minimum tendency to react with the nitrous acid system, or the acidic materials, and be compatible with the other materials in the solutions. The surface active agents, or “surfactants” may be selected from the range of available classes, but non-ionic and anionic surfactants are particularly effective. The amount of surfactant, on the final mix basis, is generally in the range of about 0.001% to about 0.10%, the level depending on the nature and effectiveness of the material in reducing the surface tension of the composition for the desired application. The instant compositions, as single-phase systems, have particular adaptability for use in aerosol form, where they may be effectively used to destroy airborne or atmospheric germs, or may be applied as sprays so as to efficiently cover contaminated surfaces. In general, preservatives will not be to stabilize the nitrous acid solutions, since the germicidal compositions will be self preserving.

When these compositions are used on human or animal skin, they may be typically applied as thickened solutions to facilitate adherence to the skin, and facilitate a greater laydown of germicide. Any thickener which is non-toxic and non-reactive with the nitrous acid system may be used. Many carbohydrate polymers are possible candidates, although some such as the cellulose-based thickeners are less preferred because of their tendency to oxidatively cleave at the β-D-glucose linkage. A preferred thickener is xanthan gum, which is minimally reactive in the nitrous acid compositions. Other appropriate thickeners include those based on poly(oxyalkylenes) and poly(acrylamides), the latter including the sulfonic acid derivatives thereof, and mineral thickeners such as the silica-based and clay gelling agents.

The amount of thickener or gelling agent which may be used in the thickened, gel composition will vary, depending upon the thickening properties of the gelling agent, the intended application, the level and nature of the acid, the level of the metal nitrite, and other additives employed. Generally, the amount may be from about 0.05 to about 30, more typically about 0.5 to about 30, preferably from about 1 to about 15, and more preferably about 1 to about 12 percent by weight of the total composition.

The amount of metal nitrite in the nitrous acid composition may be generally from about 0.01% to about 1%, typically from about 0.02% to about 0.5% and preferably from about 0.03% to 0.3% by weight. Similarly, the amount of acid, or acid-inducing salt in the composition should be sufficient such that the pH of the resulting composition will be less than about 3.75, typically from about 2.5 to about 3.6. The wide diversity of possible acid sources is such that no particular weight specification for amounts of acid is feasible except on a case-by-case basis, although the acid or material which induces an acid environment is used in the present invention in effective amounts.

The compositions of this invention may be applied to various substrates in a manner known to those skilled in this art. The compositions may be sprayed, coated or applied in any manner depending upon the substrate being treated. The compositions may be used for skin applications, for example, by applying a small but effective amount of the composition, at least five (5) minutes after preparation, to the affected area of the skin using any means known to those skilled in the art. The composition is allowed to remain on the skin, and evaporate, for a sufficient period of treatment, during which time the loss of water leads to an increased concentration of active agents resulting from the greater resulting acidity. The composition from the same container may then be reapplied periodically in order to maintain a sustained level of contact of active agents during the course of the treatment. Applications can also be made to mucosal surfaces of an animal, preferably a mammal including a human, to treat infections and inflammatory conditions in a related manner, including such areas as the cheek, the vagina, the peritoneal cavity, and internal sites exposed during surgical procedures.

The compositions may be used to disinfect surfaces, such as in medical and dental operatories and home environments. They are particularly useful in the decontamination of medical equipment, such as endoscopes and hemodialyzers, as well as related liquid pumps and dental water units. The reduced corrosion potential of the nitrous acid compositions are particularly favored where strong disinfection or sterilization of equipment is needed and where the potential for oxidation would counterindicate the use of oxidants such as chlorous acid systems. The compositions may also be used in personal hygiene formulations, such as oral rinses, toothpastes, soap formulations and douches.

The present invention is illustrated by the following Examples. All parts and percentages in the Examples, as well as the specifications and claims, are by weight, unless otherwise specified. The following examples, which are non-limiting, further describe preferred embodiments within the scope of the present invention. Many variations of these examples are possible without departing from the spirit of the invention.

### EXAMPLE 1

This example illustrates the ability of six nitrous acid solutions to destroy high levels of the Gram-positive organism Staphylococcus aureus (ATCC 29213) to a degree consistent with the relative percentage of nitrous acid with respect to total nitrite in the solution. The mixed nitrite/acid solutions, their resulting pH values, and the relative percentages of nitrous acid in the solutions were as shown below. To prepare these solutions, equal parts of a 0.625% NaNO₂ solution and increasing concentrations of malic acid solution were combined as follows:

| Sol'n No. | NaNO₂ Premix | Malic Acid Premix | Mix pH | Total Nitrite as Nitrous Acid |
|---|---|---|---|---|
| 1 | 0.625% | 2.25% | 2.94 | 70% |
| 2 | 0.625% | 1.225% | 3.12 | 60% |
| 3 | 0.625% | 0.812% | 3.35 | 47% |
| 4 | 0.625% | 0.419% | 3.54 | 37% |
| 5 | 0.625% | 0.263% | 3.75 | 28% |
| 6 | 0.625% | 0.156% | 3.90 | 21% |

Procedure: A heavy suspension of the S. aureus was prepared in saline, and 1 part of the suspension was separately combined with 10 parts of each of the above solutions, which had been prepared five minutes before the testing. After five minutes of contact, the mixtures were added to nine volumes of Dey/Engley broth to neutralize the activity and acidity. A 10-fold dilution in saline was made of this mixture. 2 mls of the sample diluted in D/E broth were added to each of five petri plates. 1 ml of the sample diluted in D/E broth was added to each of two petri plates, and 1 ml of the 1/10 dilution of the sample diluted in D/E broth was added to each of two petri plates. Approximately 10 mls of semisolid Trypticase Soy Agar were added to each petri plate, swirled and allowed to harden. The plates were incubated at 35°-37° C. for 48 hours, and the resulting colonies were enumerated.

The number of microorganisms in the original suspension was determined by making ten-fold dilutions from 10⁻¹ to 10⁻⁸. Then 1.0 ml portions of the 10⁻⁷ suspension were added to each of two sterile petri plates. 1.0 ml of the 10⁻⁸ suspension was added to each of two sterile petri plates, and 0.1 ml of the 10⁻⁸ suspension was added to each of two sterile petri plates. Approximately 10 mls of semisolid agar were added to each petri plate, swirled and allowed to harden. The plates were incubated at 35°-37° C. for 48 hours, and the resulting colonies were enumerated.

S. aureus Cidal Data*

| Sol'n No. | Recovered cfu | Log Recovery | Log Kill |
|---|---|---|---|
| 1 | 5.4 × 10¹ | 1.7 | 9.1 |
| 2 | 7.0 × 10³ | 3.8 | 7.0 |
| 3 | 4.5 × 10³ | 3.6 | 7.2 |
| 4 | 5.6 × 10⁴ | 4.7 | 6.1 |
| 5 | 6.6 × 10⁵ | 5.8 | 5.0 |
| 6 | >1 × 10⁶ | >6.0 | <4.8 |

*Inoculum suspension contained 10.8 logs of organisms.

There was significant destruction of the high inoculum of S. aureus in the 5-minute contact period, and the degree of destruction closely parallels the degree of conversion of the nitrite ion to nitrous acid. A 9.1 log kill (>1 billion-fold) was achieved with a solution in which 70% of the nitrite existed in its acidified form of nitrous acid, whereas only 5.0 logs (100,000-fold) were destroyed by the solution with nitrous acid representing 28% of the total nitrite. Even less was destroyed in the 21% nitrous acid (relative) solution.

### EXAMPLE 2

This example illustrates the ability of six nitrous acid solutions to destroy high levels of the Gram-negative organism Escherichia coli (ATCC 25922). The procedure described in Example 1 was applied in this study as well, using aliquots of the same solutions described in the Table.

The results were as follows:

Results:

E. coli Cidal Data*

| Sol'n No. | Recovered cfu | Log Recovery | Log Kill |
|---|---|---|---|
| 1 | 2.7 × 10² | 2.4 | 7.7 |
| 2 | 6.6 × 10⁴ | 4.8 | 5.3 |
| 3 | 9.0 × 10⁰ | 1.0 | 9.1 |
| 4 | 1.4 × 10¹ | 1.1 | 9.0 |
| 5 | 9.9 × 10² | 3.0 | 7.1 |
| 6 | 3.1 × 10³ | 3.5 | 6.6 |

*Inoculum suspension contained 10.1 logs of organisms.

In the case of this Gram- negative organism, the destruction of the inoculum was high in all solutions, apparently independent of pH and thus the relative amount of total nitrite existing as nitrous acid in this series of solutions. It is not known, at this point, whether this difference with respect to the observations in Example 1 is characteristic of the kill mechanism of nitrous acid solutions with respect to Gram-positive and Gram-negative organisms, or whether it relates to these particular organisms.

### EXAMPLE 3

This example illustrates the ability of six nitrous acid solutions to destroy high levels of the Gram-negative organism Escherichia coli (ATCC 25922), following 20 days of storage of the mixed solutions at ambient temperatures prior to the testing. The procedure described in Example 1 was applied in this study as well, using aliquots of the same solutions that were evaluated in Examples 1 and 2. The results were as follows:

Results:

The data are presented in the following Table, in which the kills measured on the 20-day old solutions are compared with data obtained on the T=0 mixtures (in brackets).

E. coli Cidal Data on 20-day aged mixtures*

| Sol'n No. | Recovered cfu | Log Recovery | Log Kill** |
|---|---|---|---|
| 1 | 6.0 × 10¹ | 1.8 | 9.2 [7.7] |
| 2 | 1.5 × 10² | 2.2 | 8.8 [5.3] |
| 3 | 6.0 × 10⁰ | 0.8 | 10.2 [9.1] |
| 4 | >1 × 10⁵ | >5.0 | <6.0 [9.0] |
| 5 | 3.2 × 10⁴ | 3.5 | 7.5 [7.1] |
| 6 | >1 × 10⁶ | >6.0 | <5.0 [6.6] |

*Inoculum suspension contained 11.0 logs of organisms.
**Bracketed data are log kills at T = 0 with the same solutions

About three weeks after preparation, the mixed solutions have retained a significant cidal capacity, as compared with their abilities at T=0. In fact the pH's of these aged solutions, as cf. their original values, sheds some light on the greater cidal capacity of the first few solutions tested, viz.

2.94→2.30

3.12→2.50

3.35→3.25

3.54→3.15

3.75→3.92

3.90→4.35

The highest activity, in both fresh and aged solution, appears to occur in the solutions where the pH levels dropped, leading to higher levels of nitrous acid. In these solutions, the nitrous acid and nitrite exist in a ratio of ca. 1:1 and higher. This leads to the speculation that the stability (as well as the activity) of these solutions is related to the presence of a complex ion, such as [HN₂O₄]⁻, analogous to the [Cl₂O₄]⁻ found to exist in ClO₂/ClO₂⁻ systems, where the complex [Cl₂O₄]⁻ is conjectured to be an active cidal species, of a higher oxidation potential than ClO₂ alone.

### EXAMPLE 4

This example illustrates the ability of six nitrous acid solutions to destroy high levels of the yeast Candida albicans (ATCC 10231), and to a degree consistent with the relative percentage of nitrous acid with respect to total nitrite in the solution. The mixed nitrite/acid solutions, their resulting pH values, and the relative percentages of nitrous acid in the solutions were similar to those shown in Example 1.

Procedure: A heavy suspension of the C. albicans was prepared in saline, and 1 part of the suspension was separately combined with 10 parts of each of the above solutions, which had been prepared five minutes before the testing. After five minutes of contact, the mixtures were added to nine volumes of Dey/Engley broth to neutralize the activity and acidity. A 10-fold dilution in saline was made of this mixture. 2 mls of the sample diluted in D/E broth were added to each of five petri plates. 1 ml of the sample diluted in D/E broth was added to each of two petri plates, and 1 ml of the 1/10 dilution of the sample diluted in D/E broth was added to each of two petri plates. Approximately 10 mls of semisolid Sabouraud Dextrose Agar were added to each petri plate, swirled and allowed to harden. The plates were incubated at 20°-25° C. for 72 hours, and the resulting colonies were enumerated.

The number of microorganisms in the original suspension was determined by making ten-fold dilutions from 10⁻¹ to 10⁻⁸. Then 1.0 ml portions of the 10⁻⁷ suspension were added to each of two sterile petri plates. 1.0 ml of the 10⁻⁸ suspension was added to each of two sterile petri plates, and 0.1 ml of the 10⁻⁸ suspension was added to each of two sterile petri plates. Approximately 10 mls of semisolid agar were added to each petri plate, swirled and allowed to harden. The plates were incubated at 20°-25° C. for 72 hours, and the resulting colonies were enumerated.

C. albicans Cidal Data*

| Sol'n No. | Recovered cfu | Log Recovery | Log Kill | % HONO** |
|---|---|---|---|---|
| 1 | 0 | 0 | >7.86 | 70 |
| 2 | 4 | 0.6 | 7.26 | 60 |
| 3 | 2.4 × 10¹ | 1.38 | 6.48 | 47 |
| 4 | 2.1 × 10⁴ | 4.32 | 3.54 | 37 |
| 5 | >1 × 10⁶ | >6 | <˜1 | 28 |
| 6 | >1 × 10⁶ | >6 | <˜1 | 21 |

*Inoculum suspension contained 7.86 logs of organisms.
**% of total nitrite ion present as nitrous acid

The destruction of the C. albicans yeast is quite significant, particularly for the solutions below about 3.5, where the nitrous acid is present in a ratio of about 1:1 with respect to ionic nitrite (i.e. above about 50% of total nitrite as HONO). Thereafter the fall off in kill is rather dramatic, at higher pHs. For this organism, as for the S. aureus of Example 1, this suggests that a 1:1 adduct of nitrous acid and nitrite may be providing particularly effective cidal capacity in this system.

### EXAMPLE 5

This example illustrates the ability of six nitrous acid solutions to destroy high levels of the mold Aspergillus niger (ATCC 6275). The mixed nitrite/acid solutions, their resulting pH values, and the relative percentages of nitrous acid in the solutions were similar to those shown in Example 1, and the procedure followed paralleled that provided in Example 4.

A. niger Cidal Data*

| Sol'n No. | Recovered cfu | Log Recovery | Log Kill |
|---|---|---|---|
| 1 | 18 | 1.26 | 7.14 |
| 2 | 83 | 1.92 | 6.48 |
| 3 | 30 | 1.48 | 6.92 |
| 4 | 37 | 1.57 | 6.83 |
| 5 | 0 | 0 | >8.40 |
| 6 | 0 | 0 | >8.40 |

*Inoculum suspension contained 8.40 logs of organisms.

### EXAMPLE 6

This example illustrates the high level and duration of efficacy of a nitrous acid teat dip composition against the Environmental organism E. Coli (ATCC 25922). An in vitro microbiological evaluation was run on the composition at three times; when freshly mixed as well as 1 day and 2 days after preparation. The two components of the teat dip were as follows:

| | | |
|---|---|---|
| Nitrite Base: | Sodium nitrite- | 0.625% |
| | Sodium dodecylbenzene sulfonate- | 0.20% |
| | FD&C Yellow #5- | 0.20% |
| | Water- | q.s. |
| Acid Activator: | Lactic acid (88%)*- | 3.23% |
| | Glycerin- | 10.0% |
| | Natrosol 250MR- | 0.50% |
| | Sodium benzoate- | 0.04% |
| | Benzalkonium chloride (17%) | 1.26% |
| | Water- | q.s. |

*HCl was added so that a 1:1 mix of both parts had a pH of 2.95.

Procedure: The initial inoculum at each test period was >10⁸, as will be seen in the test data. The microorganism was plated on Trypticase Soy Agar and incubated at 35°-37° C. for 24 hours. A heavy suspension was prepared in sterile saline. Equal quantities (by weight) of the teat dip components were mixed together, and allowed to stand for about 10 minutes. Then nine volumes of this sample was challenged with one volume of the organism suspension for 15 seconds. Then 2.0 ml of the mixture were added to 18 ml of D/E broth. A further 1/10 dilution of the D/E broth in saline was prepared. Five 2.0 ml samples of the D/E broth were added to petri plates. Duplicate 1.0 ml samples were added to petri plates, and duplicate 1.0 ml samples of the 1/10 dilution were added to petri plates. Approximately 10 ml of liquid Trypticase Soy Agar were added to each petri plate and allowed to solidify. Plates were incubated at 35°-37° C. for 24-48 hours, and colony forming units were counted. Thereafter the mixed sample was incubated in a foil-covered sterile container at room temperature, until use. After the first sample (Day 0) sample was tested, samples were removed for testing 1 and 2 days after mixing (Day 1 and 2, resp.) and were tested as above. At each test point a control study was run, in which a sample of saline was challenged, instead of the test sample.

Results:

| | | | |
|---|---|---|---|
| | Challenge Inoculum | Organisms Recovered | Log |
| Test Sample | (Log #cfu/ml Product) | (Log #cfu/ml Product) | Reduction |

Day 0

| | | | |
|---|---|---|---|
| Teat Dip | 7.8 × 10⁸ (8.89) | 0 | >8.89 |
| Control | 7.8 × 10⁸ (8.89) | 3.8 × 10⁸ | |
| (Saline) | | | |

Day 1

| | | | |
|---|---|---|---|
| Teat Dip | 5.3 × 10⁸ (8.72) | 1.7 × 10¹ (1.23) | 7.49 |
| Control | 5.3 × 10⁸ (8.72) | 5.1 × 10⁸ | |
| (Saline) | | | |

Day 2

| | | | |
|---|---|---|---|
| Teat Dip | 3.4 × 10⁸ (8.53) | 0 | >8.53 |
| Control | 3.4 × 10⁸ (8.53) | 3.6 × 10⁸ | |
| (Saline) | | | |

These results clearly demonstrate that the nitrous acid teat dip was capable of destroying upwards of 100 million E. coli organisms within 15 seconds of contact, up through two days following mixture. The 17 remaining organisms, of the 530 million challenge at Day 1, are considered artifactual, in as much as the 2-day aged sample destroyed all of the challenge. It is evident from these data that nitrous acid antimicrobials can exert continued cidal activity against mastitis-causing microorganisms long after their initial preparation.

### EXAMPLE 7

This example illustrates the prolonged high-level efficacy of a thickened version of the above nitrous acid teat dip composition against the Environmental organism E. coli (ATCC 25922). This type of teat dip is generally termed a “barrier” dip, because it deposits a protective film on the teat during and after drying, so as to protect the teat during the intermilking period. The composition provided in Example 6 was modified by the addition of two components to the nitrite base, specifically 0.50% xanthan gum and 2.24% of Fixomer A-30, a 70/30 copolymer of methacrylic acid and poly(acrylamidomethyl propane sulfonic acid). In this study, in vitro microbiological evaluations were run on the composition at five times; when freshly mixed (i.e., 10-minutes after combination) as well as 1, 2, 6 and 14 days after preparation. The procedure was the same as in Example 6, except that a 1-minute contact was used for the studies, based on the extended contact of a barrier dip, which is applied post-milking, and intended to last on the teat for up to ˜12 hours until the next milking.

Results:

| | | | |
|---|---|---|---|
| | | Organisms | |
| | Challenge Inoculum | Recovered (Log | Log |
| Test Sample | (Log #cfu/ml Product) | #cfu/ml Product | Reduction |

Day 0

| | | | |
|---|---|---|---|
| Teat Dip | 2.2 × 10⁸ (8.34) | 0 | >8.34 |
| Control (Saline) | 2.2 × 10⁸ (8.34) | 2.5 × 10⁸ | |

Day 1

| | | | |
|---|---|---|---|
| Teat Dip | 4.0 × 10⁸ (8.60) | 1.7 × 10¹ | >8.60 |
| Control (Saline) | 4.0 × 10⁸ (8.60) | 2.7 × 10⁸ | |

Day 2

| | | | |
|---|---|---|---|
| Teat Dip | 3.4 × 10⁸ (8.53) | 0 | >8.53 |
| Control (Saline) | 3.4 × 10⁸ (8.53) | 2.4 × 10⁸ | |

Day 6

| | | | |
|---|---|---|---|
| Teat Dip | 3.2 × 10⁸ (8.51) | 0 | >8.51 |
| Control (Saline) | 3.2 × 10⁸ (8.51) | 4.1 × 10⁸ | |

Day 14

| | | | |
|---|---|---|---|
| Teat Dip | 7.8 × 10⁸ (8.89) | 0 | >8.89 |
| Control (Saline) | 7.8 × 10⁸ (8.89) | 3.8 × 10⁸ | |

These results clearly demonstrate that the nitrous acid barrier teat dip was capable of destroying 220-780 million E. coli organisms within 60 seconds of contact, up through two weeks following mixture. As demonstrated in Example 6, and further evident from these data, nitrous acid teat dips can exert continued and very high cidal activity against mastitis-causing microorganisms long after the teat dip's initial preparation.

### EXAMPLE 8

This example illustrates the ability of one of the six nitrous acid solutions tested in Examples 1, 2, and 3, specifically Solution No. 2, to be as microbiocidally effective after over two (2) years of storage at ambient temperatures, as it was in both Example 2 (the day of preparation) and Example 3 (after 20 days of ambient storage). In Example 2 the nitrous acid, formulated with equal parts of 0.625% NaNO₂ and 1.225% Malic Acid, was shown to destroy 5.3 logs of the Gram-negative organism Escherichia coli (ATCC 25922) after 5 minutes of contact. In Example 3, after 20 days of storage, the aged solution destroyed 8.8 logs of that organism.

After over 26 months of ambient storage (specifically 735 days), an aliquot of that nitrous acid solution was tested for its 5-minute kill, with the following results:

Test Organism: E. coli ATCC 25922
Initial Suspension: 1.4 × 10⁹

| | Challenge | | |
|---|---|---|---|
| | Inoculum | Recovered | Log |
| Test Sample | (Log cfu/ml) | (Log cfu/ml) | Reduction |
| Sample mixed on Sep. 28,2001 | 1.4 × 10⁸ | 0 | >8.15 |
| and stored at room temp. | (8.15 logs) | | |
| Control (Saline) | 1.4 × 10⁸ | 1.1 × 10⁸ | — |

The procedure for E. coli was the same as described in the earlier Examples as follows:

The microorganism was plated on Trypticase Soy Agar and incubated at 35-37° C., for 18-24 hours. A heavy suspension was prepared in sterile saline. The challenge sample, which had been mixed on Sep. 28, 2001, had been stored in a capped glass test tube at room temperature until testing. Nine volumes of the sample (1.8 ml) were challenged with one volume (0.2 ml) of the organism for 5 minutes. Following this 2.0 ml of this mixture was added to 18 ml of D/E broth. A further 1/10 dilution of the D/E broth in saline was prepared. Five 2.0 ml samples of the D/E broth were added to petri plates. Duplicate 1.0 ml samples were added to petri plates, and duplicate 1.0 ml samples of the 1/10 dilution were added to petri plates. Approximately 10 ml of liquid Trypticase Soy Agar was added to each petri plate and allowed to solidify. Plates were incubated at 35-37° C., for 24 hours, and colony forming units were counted. A control study was run, in which a sample of saline was challenged, instead of the test sample.

This Example clearly demonstrates that this nitrous acid solution, at a pH below about 3.4 (as deduced from the aging data in Example 3 and the pH information provided in Example 1), is capable of providing a high level of antimicrobial activity, for at least several years after its formation, when stored under ambient conditions.

Based on the foregoing, it is clear that the present invention is well adapted to carry out the objects, and achieve the ends and advantages mentioned at the outset. While currently preferred embodiments of the invention have been described for purposes of this disclosure, numerous modifications may be made which will readily suggest themselves to those skilled in the art, and which are encompassed within the spirit of he invention disclosed, and as defined in the appended claims.

What is claimed is:

1. A composition comprising a single-phase liquid or gel comprising nitrous acid and an alpha hydroxyl acid or phosphoric acid, wherein:
(a) the pH of the composition either remains relatively constant at an initial value of around 3.75 or lower, or decreases from said initial value of around 3.75 or lower at the time of formulation to a value as low as around 2.5 over a period of at least about two days, preferably about two days to five days;
(b) the molar percentage of nitrite ion in the composition in the form of nitrous acid is greater than about 35% but less than about 95% of the total nitrite ions present in the composition; and
(c) the composition exhibits cidal activity against microorganisms for a period of at least two months after formulation.

2. A composition of claim 1, wherein the composition comprises a compound comprising an amount of phosphoric acid with a pKa of about 2.15 that is sufficient to lower the pH of the composition to less than about 3.75.

3. A composition of claim 1, wherein the alpha hydroxyl acid is a compound of the formula (I): 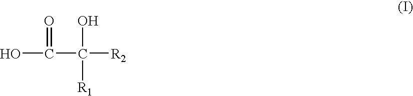
wherein R₁and R₂may be the same or different and may be selected from the group consisting of hydrogen, methyl, —CH₂ COOH, —CH₂ COO⁻, —CH₂ OH, —CHOHCOOH, —C₆H₅, and —CH₂ C₆H₅.

4. A composition of claim 1, wherein the composition further comprises one or more of the following: a surface active material, a chelating agent, an effervescent compound, and a thickener.

5. A composition of claim 1, wherein the cidal activity of the composition over a period of about twenty-four months or more after formulation is comparable to the activity that it demonstrated initially.

6. A composition of claim 1, wherein the cidal activity of the composition over a period of about five minutes or more after formulation is equivalent to the activity necessary to achieve an approximately eight log decrease in a sample of E. coli.

7. A composition of claim 1, wherein the composition is used in conjunction with an application medium.

8. A composition of claim 1, wherein the nitrous acid is generated by a metal nitrite.

9. A composition of claim 1, wherein the composition is a liquid teat dip.

10. A composition of claim 1, wherein the composition is a gel.

11. A method comprising disinfecting a substrate by application of a composition of claim 1.

12. A method of claim 1, wherein the substrate is mammalian tissue.

13. A method of claim 1, wherein the substrate is a metal surface.

14. A composition of claim 1, comprising an amount of nitrite in the form of nitrous acid that is no more than about 85% by weight of the total nitrite ions in the composition.

15. A composition of claim 1, wherein the composition is a disinfecting gel comprising a thickener.

16. A composition of claim 1, wherein the composition is an oral rinse.

17. A method comprising maintaining disinfection of a substrate over a period of at least around several months by applying an effective amount of a composition of claim 1 to the substrate.

18. A method of claim 17, wherein the substrate is mammalian tissue.

19. A method of claim 17, wherein the substrate is a metal surface.

20. A composition of claim 1, wherein the composition may be sprayed onto a substrate.
